(19) United States
(12) Patent Application Publication
(10) Pub. No.: US 2019/0104937 A1
(43) Pub. Date: Apr. 11, 2019

(54) Vestibulogram

(71) Applicant: Massachusetts Eye and Ear Infirmary, Boston, MA (US)

(72) Inventor: Daniel Michael Merfeld, Lincoln, MA (US)

(21) Appl. No.: 16/056,283

(22) Filed: Aug. 6, 2018

Related U.S. Application Data
(63) Continuation of application No. 12/990,535, filed on Nov. 16, 2010, now abandoned.

(51) Int. Cl. A61B 5/00; A61H 1/00

(52) U.S. Cl. CPC A61B 5/00; A61B 5/4023; A61B 3/113; A61B 5/7475; A61B 5/4884; A61H 1/001

(57) ABSTRACT

An apparatus for determining a subject's threshold for perceiving acceleration includes a motion platform to execute motions and to receive a response to the executed motions from a subject on the motion platform and a feedback system in communication with the motion platform to receive the subject's responses and to determine a next motion. The next motion has at least one feature determined based on the response of the subject to the executed motions. The apparatus also includes a controller connected to the motion platform and the feedback system to cause the motion platform to execute the motions defined by a motion set

### CROSS-REFERENCE TO RELATED APPLICATIONS

This application is a continuation of U.S. patent application Ser. No. 12/990,535, filed Nov. 16, 2010, which is a National Stage of International No. PCT/US2008/063158, filed on May 9, 2008, the contents of which are hereby incorporated by reference in its entirety.

### TECHNICAL FIELD

This invention relates to the vestibular system, and in particular, to the diagnosis of vestibular dysfunction.

### BACKGROUND

The vestibular system of the inner ear enables one to perceive body position and movement. In an effort to assess the integrity of the vestibular system, it is often useful to test its performance. Such tests are often carried out at a vestibular clinic.

Vestibular clinics typically measure reflexive responses like balance or the vestibulo-ocular reflex to diagnose a subject's vestibular system. The vestibulo-ocular reflex is one in which the eyes rotate in an attempt to stabilize an image on the retina. Since the magnitude and direction of the eye rotation depend on the signal provided by the vestibular system, observations of eye rotation provide a basis for inferring the state of the vestibular system.

Measurements of eye movement are useful for diagnosing some failures of the vestibular system. However, some patients report perceptual vestibular problems and still test normal on standard diagnostic tests that assess the vestibulo-ocular reflex. This demonstrates that current diagnostic techniques do not adequately assess all aspects of vestibular function, especially perceptual aspects.

The failure of vestibulo-ocular reflex measurement might result because reflexive vestibular responses and vestibular perception use different neural pathways. The failure may also arise because some disorders involve subtleties that are not assessed by measuring the vestibulo-ocular reflex. For example, vestibulo-ocular reflex tests typically assess responses to motions with relatively large amplitudes. But it may also be important to conduct tests having motions with smaller amplitudes.

Therefore, existing devices and methods fail to assess and/or characterize perceptual responses evoked by vestibular stimulation, particularly in clinical settings.

### SUMMARY

In one aspect, the invention features an apparatus for determining a subject's threshold for perceiving acceleration. The apparatus includes a motion platform to execute motions and to receive a response to the executed motions from a subject on the motion platform and a feedback system in communication with the motion platform to receive the subject's responses, and to determine a next motion. The next motion has at least one feature determined based on the response of the subject to the executed motions. The apparatus also includes a controller connected to the motion platform and the feedback system to cause the motion platform to execute the motions defined by a motion set.

In another aspect, the invention features a computer-readable medium having encoded thereon software for determining a subject's threshold for perceiving acceleration. The software includes instructions for causing a data processing system to carry out the steps of (a) commencing execution of a first motion set defining a sequence of motions; (b) recording responses of the subject to the motions in the sequence of motions defined by the first motion set; (c) determining that the responses of the subject satisfy a condition; (d) terminating execution of the first motion set; (e) at least in part on the basis of the subject's responses to the motions in the sequence of motions defined by the first motion set, defining a second motion set defining a sequence of motions; (f) commencing execution of the second motion set; and (g) recording responses of the subject to the motions in the sequence of motions defined by the second motion set.

In another aspect, the invention features a method for determining a subject's threshold for perceiving a motion. The method includes steps: (a) commencing execution of a first motion set defining a sequence of motions; (b) recording responses of the subject to the motions in the sequence of motions defined by the first motion set; (c) determining that the responses of the subject satisfy a condition; (d) terminating execution of the first motion set; (e) at least in part on the basis of the subject's responses to the motions in the sequence of motions defined by the first motion set, defining a second motion set defining a sequence of motions; (f) commencing execution of the second motion set; and (g) recording responses of the subject to the motions in the sequence of motions defined by the second motion set.

Embodiments may include one or more of the following features. The controller can be configured to cause the motion platform to execute a sinusoidal acceleration. The motion set can define an acceleration having a maximum magnitude and the feature includes the maximum magnitude. The feature can include a direction of the motion. Each motion can belong to a type selected from the group consisting of translation, rotation, and a combination of translation and rotation. The feedback system can detect the subject's vestibulo-ocular reflex.

Embodiments may also include one or more of the following features. The method can also include determining a maximum magnitude and frequency for each motion of the first motion set before commencing execution of a first motion set. Determining that the responses of the subject satisfy a condition can include determining how many recorded responses are correct and comparing the number of correct responses to a threshold. Defining the second motion set can include determining a maximum magnitude and frequency for each motion of the second motion set based on the recorded responses. The method can also include examining the responses of the subject to the motions defined by the first motion set and determining the subject's threshold before commencing execution of the second motion set. The method can also include, after step (g), defining the second motion set to be a new first motion set, and repeating the steps (c) to (d). The method can also include determining a motion threshold on the basis of the responses of the subject; selecting a new frequency; and repeating the steps (a) to (g) for determining thresholds corresponding to the new frequency. Recording responses of the subject can include observing the subject's vestibulo-ocular reflex.

All publications, patent applications, patents, and other references mentioned herein are incorporated by reference herein in their entirety.

Other features and advantages of the invention will be apparent from the following detailed description, from the claims, and from the drawings, in which:

### DESCRIPTION OF DRAWINGS

FIG. 1 is a schematic diagram of a vestibular test system;

FIG. 2 is a schematic diagram of a motion platform in the vestibular test system of FIG. 1;

FIG. 3 is a flow chart exemplifying a method of a vestibular test using the vestibular system of FIG. 1;

FIG. 4 is a motion list describing a motion set;

FIG. 5 is a portion of a feedback list;

FIG. 6 shows a time series of acceleration amplitude A;

FIG. 7 shows a motion package;

FIG. 8 shows a portion of statistical results; and

Like reference symbols in the various drawings indicate like elements.

### DETAILED DESCRIPTION

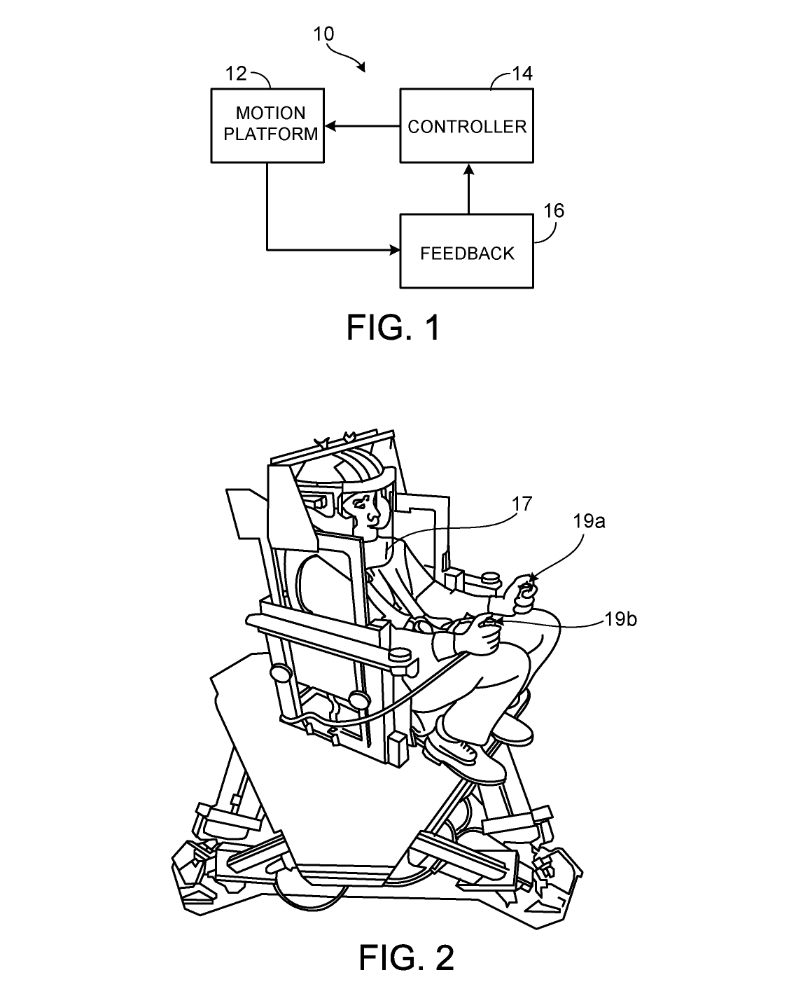

Referring to FIG. 1, a vestibular test system 10 includes a motion platform 12, a controller 14, and a feedback system 16.

The motion platform 12 holds a subject whose vestibular system is to be tested, and moves that subject in response to instructions from the controller 14. Generally, each motion provided by the motion platform 12 is described by a motion profile that includes information about the direction of motion and other features related to the motion. For example, a motion can be a translational motion along any of the three perpendicular axes x, y, and z of a coordinate system centered on the motion platform 12. Generally, the z axis is vertical to the ground on which the motion platform 12 rests, and the x and y axes define a plane parallel to the ground. In some embodiments, the motion platform 12 is arranged so that the subject is oriented with his spine parallel to the z axis.

The motion platform 12 can also provide various rotational motions such as a roll, which is a rotation about the x axis; a pitch, which is a rotation about they axis; and a yaw, which is a rotation about the z axis.

In some embodiments, the motion profile includes the amplitude and frequency of the velocity and acceleration of the motion. The amplitude of the acceleration and velocity vary with time, whereas the frequency remains constant. For example, a translational motion starts with a zero velocity, accelerates to a maximum velocity, and decelerates to zero again. In some embodiments, the acceleration is sinusoidal and can be expressed as

a(t)=A sin(2πft) (1)

where a(t) is the acceleration at time t, A is the acceleration amplitude, and f is the frequency. With such an acceleration, starting from zero, the translational velocity v(t) at time t is

v(t)=A/(2πf)[1−cos(2πft)] (2)

Similarly, a rotational motion can include a sinusoidal angular acceleration and an angular velocity, both of which are expressed in a manner similar to the translational acceleration and velocity of equations (1) and (2).

Referring to FIG. 2, a motion platform 12 moves a subject 17 seated thereon according to motion profiles, as discussed above. A suitable motion platform 12 is a MOOG® series 6DOF2000E (manufactured by Moog Inc., East Aurora, N.Y.).

The motion platform 12 includes an actuator to allow the subject 17 to unambiguously communicate his perception of motion. A suitable actuator is a pair of buttons 19a, 19b. However, the actuator can also be a joystick, a pair of joysticks, a pair of switches, or even foot pedals.

In some embodiments, the subject 17 communicates his perception of motion to an operator by speaking or by gesture. In such cases, the operator provides a suitable input to an actuator, for example by using a keyboard to record data indicative of the subject's perception of motion.

During a test, the subject 17 presses one of the buttons 19a, 19b to indicate that he perceives motion. The particular button pressed indicates the subject's perception of the motion's direction. The number of times that the subject 17 presses the correct button 19 provides a basis for assessing his vestibular system. To avoid confusing the vestibular system with false signals caused by the subject's own head motion, an adjustable head brace 21 holds the subject's head in place such that it is bisected by the xz plane.

Referring back to FIG. 1, when the subject 17 presses a button 19a, the feedback system 16 transmits a corresponding response signal to the controller 14. In some embodiments, the feedback system 16 includes two buttons. The subject can press either button to indicate his perception of the motion he is subjected to.

The choice of which button to press and when to press it is based on a pre-agreed rule between the subject 17 and a test operator. For example, the subject can agree to press the first button upon perceiving an upward translational motion and to press the second button upon perceiving a downward translational motion. The rule can be adjusted upon agreement between the subject and the test operator.

The buttons 19a, 19b are electronically connected to a processor that records, on a feedback list 28, which buttons were pressed and what motion was occurring at the time. Periodically, the processor examines the feedback list 28 and causes the controller 14 to determine the motion profile of the next motion to be applied to the subject 17. In this way, the vestibular test system 10 automatically and adaptively changes motion profiles in response to the subject's perception of motion.

The processor determines the motion profile parameters, such as acceleration amplitude A, velocity amplitude A/πf, frequency f, and direction, on the basis of the feedback list 28. In response to instructions from the processor, the controller 14 then causes the motion platform 12 to move in a manner consistent with the determined motion profile. In some embodiments, the controller 14 includes a microprocessor or a computer.

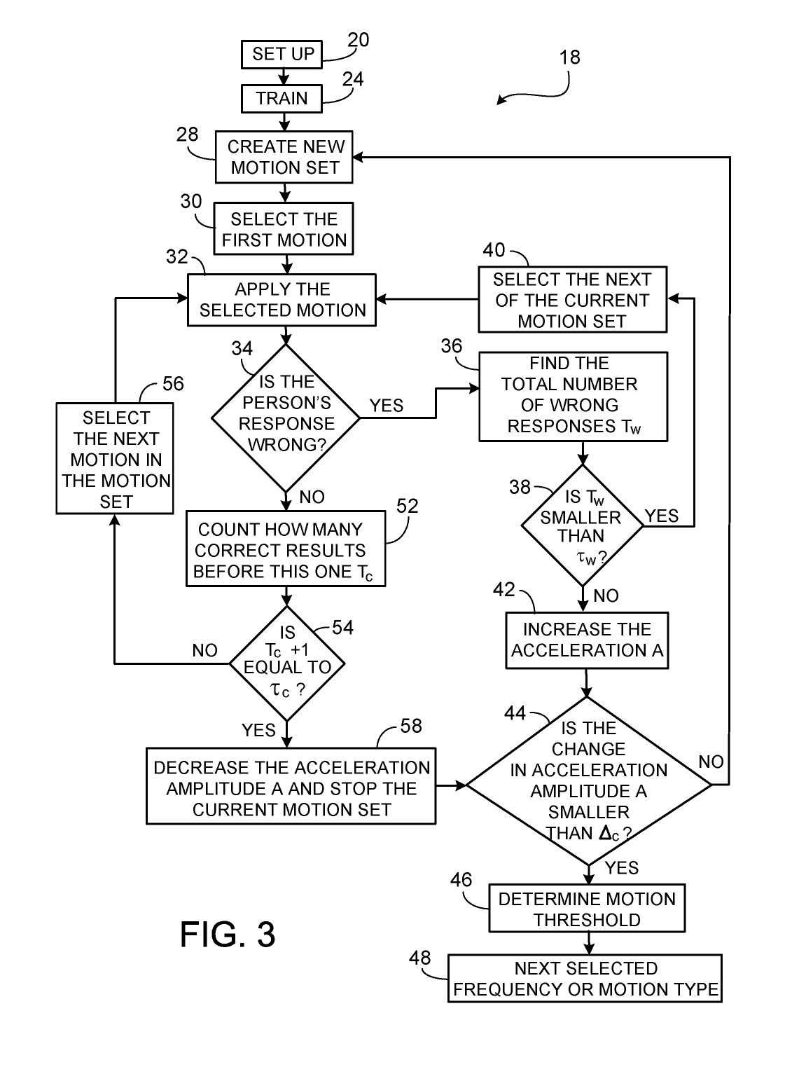

Referring to FIG. 3, a vestibular test 18 using the vestibular test system 10 in FIG. 1 determines a subject's motion threshold by moving the subject in a manner defined by the motion set. Each motion set includes several motions, each characterized by a motion profile. In the embodiment described herein, each motion profile characterizes a sinusoidal acceleration with a uniform frequency f and acceleration amplitude A. The subject's motion threshold at a particular frequency f is the acceleration amplitude A at which the subject perceives motion at that frequency.

The vestibular test 18 starts with a set-up step 20 in which the subject 17 is seated and stabilized on the motion platform 12 at the initial position.

The setup step 20 is followed by a training step 24, the purpose of which is to enhance the likelihood that the subject accurately communicates his perception of motion by pressing the correct button 19. The training step 24 includes trial tests to train the subject 17 to press the correct button in response to perceiving a particular motion. To assist the process, the training step 24 can be performed in a lighted room to enable the subject 17 to use vision to help perceive the motion.

To minimize the influence of non-vestibular cues regarding motion direction, the remaining steps of the vestibular test 18 are performed without any visual cues indicating the motion. For example, the movement may be applied in the dark or with a visual display, e.g., a small light emitting diode, that moves with the subject during any motion. To reduce reliance on wind cues, the subject's skin surfaces are covered, for example, with long sleeves, or gloves, and a visor is attached to the head holder 21. To reduce reliance on audio cues, the subject 17 is provided with earplugs to reduce the external noise by about 20 dB, and exposed to white noise (circa 60 dB). Reliance on tactile cues is minimized by evenly distributing pressure via padding.

Multiple motion sets are provided to the subject 17 to obtain a motion threshold for the subject 17 at a selected frequency f. In one embodiment, each motion set includes motions of the same type. For example, a motion set would include nothing but yaw motions, or nothing but pitch motions.

Referring to FIG. 4, a motion set 26 is described by a list of motion profiles 50a-e, which defines a sequence of motions. In FIG. 4, there are five motion profiles 50a-e corresponding to five translations along the x axis. Each motion profile 50a-e is characterized by a direction, an acceleration amplitude A, and the selected frequency f. In some embodiments, all motion profiles 50a-e in the motion set 26 have the same acceleration amplitude A and frequency f, but each has a random direction. For example, the first motion profile 50a of the motion set 26 is a translation in the positive direction of the x axis, and is labeled as “+x.” The second motion profile 50b is a translation in the negative direction along the x axis, and is labeled as “−x”. The direction, “+x” or “−x”, for each motion profile 50a-e of the motion set 26 is randomly assigned.

The acceleration amplitude A is determined based on the subject's responses to previous motion sets 26 applied to the subject 17. For example, when the motion set 26 is the first motion set to be applied to the subject 17 along a particular axis or about a particular axis, the acceleration amplitude A is chosen to be about 10 to 20 times larger than the motion threshold of a normal subject for that particular type of motion.

When the motion set 26 is not the first motion set to be applied to the subject 17, the acceleration amplitude A is determined based on the previous motion sets applied. The motion profiles 50a-e comprising a motion set 26 are determined prior to applying, to the subject 17, the motions specified in the motion set 26.

Typically, before beginning each motion, an alert is sent, for example, in the form of a tone or light, to the subject 17 to notify the subject that a motion is going to start. In some embodiments, after each motion ends, another alert is sent to the subject 17 to notify the subject 17 that a response is now required if the subject has not responded already. In cases where the acceleration amplitude was too low for the subject 17 to detect any motion, this response would essentially be a guess.

The response of the subject 17 to each motion of the multiple motion sets, together with the motion's direction, for example, “+x” for positive direction of x axis and “−x” for the negative direction as described in FIG. 4, acceleration amplitude A and frequency f are recorded in a feedback list 28, as shown in FIG. 5. The result of the subject's perception of each motion can be marked in the form of numbers, letters, or symbols, for example, “0” for failure and “1” for correct perception.

Referring back to FIG. 3, following creation of a motion set (step 27), the controller selects the first motion profile of that motion set (step 30) and causes the motion platform 12 to execute the motion defined by that motion profile (step 32). This motion set is referred to as the “incumbent motion set.”

If the subject's perception is correct (step 34), the feedback list, in which the subject's responses have been accumulated, is inspected to determine the total number T_{c} of correct results for the incumbent motion set (step 53). If the total number (T_{c}) of correct results is less than a threshold τ_{c} (step 55), then the next motion in the incumbent motion set is selected (step 57) and applied to the subject (step 32).

If T_{c} is equal to the threshold τ_{c}, then the test is too easy for the subject, and no further motions are selected from the incumbent motion set. As a result, in some cases execution of the motion set is terminated prior to completing the sequence of motions defined by the motion set. Instead of completing the sequence of motions, a decreased acceleration amplitude A⁻ is calculated (step 58) to make the test more difficult. This process is repeated until a subject provides an incorrect response.

In one embodiment, the decreased acceleration amplitude will be 50% of the acceleration amplitude at which the total number of correct responses reached the threshold. For other embodiments, the decreased acceleration amplitude might be some other fixed percentage (e.g., 75%) of the most recent acceleration amplitude at which the total number of correct responses reached the threshold.

In yet other embodiments, the reduction in the acceleration amplitude may equal 50% of the most recent increase in the acceleration amplitude. Other embodiments may decrease the acceleration amplitudes by other percentages (e.g., 25%) of the most recent increase in the acceleration amplitude.

If, on the other hand, the subject's response is wrong (step 34), the feedback list is inspected to determine the total number T_{w} of wrong results recorded thus far for the incumbent motion set (step 36).

If T_{w} is less than a threshold τ_{w} (step 38), then the next motion profile in the motion set is selected (step 40) and its corresponding motion applied to the subject (step 32). If T_{w} is equal to the threshold τ_{w}, the test is assumed to be too difficult for the subject, and no further motion profiles are selected from the incumbent motion set. Instead, to make the test easier, an increased acceleration amplitude A⁺ is calculated (step 42). As a result, in some cases, execution of the sequence of motions is terminated prior to completion of all motions in the motion set.

In some embodiments, the increase in the acceleration amplitude might equal some percentage (e.g., 50%, 60%, 70%, etc.) of the difference between the current acceleration amplitude and the acceleration amplitude at which the subject last correctly identified the direction of motion.

The test can terminate if the difference ΔA between the original acceleration amplitude A and either the decreased acceleration amplitude A⁻ or the increased acceleration amplitude A⁺ is smaller than a threshold Δc (step 44). When this occurs, the vestibular test is regarded as having been completed for the selected frequency and type of motion, and the motion threshold for the subject is determined on the basis of the acceleration amplitude, for example by evaluating the mean of the original amplitude (step 46) and either the increased or decreased amplitude. The vestibular test then proceeds to a new selected frequency and/or motion type (step 48).

Alternatively, the test can terminate upon occurrence of a pre-defined number of local minima in the sequence of acceleration amplitudes used during the test.

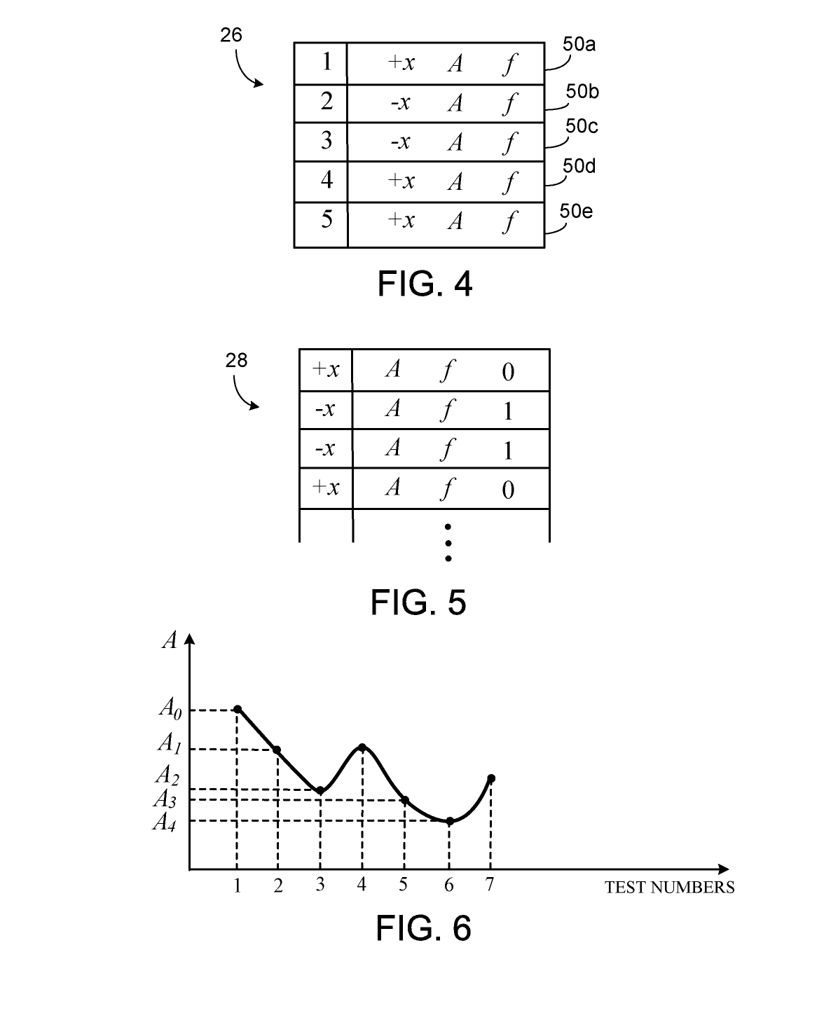

Referring to FIG. 6, the acceleration amplitude applied to the subject 17 varies depending on whether the subject 17 responds correctly or incorrectly. In general, when the subject 17 responds correctly, the amplitude decreases; and when the subject responds incorrectly, the amplitude increases. As the test progresses, the resulting time series of acceleration amplitudes naturally develops maxima and minima. For example, the particular time series shown in FIG. 6 features two local minima, one at the third motion test, the other at the sixth motion test.

As is apparent from FIG. 6, the subject 17 responded correctly to the tests in which the acceleration amplitude progressively decreases from A₀ to A₁ and responded incorrectly when the acceleration amplitude was further reduced to A₂. The subject 17 further responded correctly to tests with acceleration amplitudes A₃>A₁ and failed the next test with acceleration amplitude A₄. A local minimum in the time series is thus formed each time a subject responds incorrectly after having responded correctly two or more times.

In one embodiment, the test would terminate if the acceleration amplitude yielding the incorrect response was the 2^{nd} local minimum in the sequence of acceleration amplitudes tested. Other embodiments might terminate at the 3^{rd} local minimum or the 4^{th} local minimum.

In an alternative embodiment, the increased acceleration amplitude would be an acceleration amplitude at which the subject most recently attained a particular score, i.e., a particular number of correct answers. By using this acceleration more than once, one can average out the effects of noise or other variations that may otherwise corrupt the test results. In such an embodiment, the test 18 skips the step 44 of FIG. 3 and continues with step 27 directly. To complete the test 18, each acceleration amplitude is used in, for example, less than two, three, or four motion sets.

The method described in FIG. 3 can be used to systematically determine other motion thresholds at different frequencies for the same or other types of motions, including translation, pitch, roll, yaw, or a combination thereof. For each type of motion, the relationship between the motion thresholds and the motion frequency f defines a vestibulogram for the subject 17. Because of the systematic manner in which the method acquires such information, and the minimal intervention required, the method is particularly suited for clinical use. Moreover, the method described herein can be used to efficiently collect data to create a graph of motion threshold as a function of frequency, referred to herein as a “vestibulogram,” for each of several motion directions.

The determination or calculation of the increased acceleration amplitudes A⁺ and A⁻ can vary. In some embodiments, when the feedback list 28 indicates that the subject 17 has yet to experience an acceleration amplitude greater than the last amplitude A, A⁺ is set to be, for example, 60%, 50%, 40%, 30%, or 20%, greater than A. In other cases, the feedback list 28 indicates that the subject 17 has experienced an acceleration amplitude A₀ that is greater than the most recently used acceleration amplitude A but less than all other acceleration amplitudes recorded on the feedback list 28. When this occurs, the increased acceleration amplitude A⁺ is set to be between A and A₀, for example, 60%, 50%, 40%, 30%, or 20% of (A₀−A) greater than A.

Conversely, when the feedback list 28 indicates that the subject 17 has yet to experience an acceleration amplitude less than the most recent acceleration amplitude A, A⁻ is set to be, for example, 60%, 50%, 40%, 30%, or 20%, of the most recent acceleration amplitude A. In some cases, the feedback list 28 includes an acceleration amplitude A₀ that is less than the most recent acceleration amplitude A but greater than all other acceleration amplitudes recorded on the feedback list 28. When this occurs, the decreased acceleration amplitude A⁻ is set to be between A and A₀, for example, 60%, 50%, 40%, 30%, or 20% of (A−A₀) less than A.

The thresholds τ_{w}, τ_{c}, and Δc can also vary with different embodiments. For example, there exist embodiments in which τ_{w} is 1, 2, or 3. There also exist embodiments in which τ_{c} is 2, 3, or 4. Additional embodiments include those in which Δc is 5%, 4%, or 3% of the last acceleration amplitude A. However, these are by no means the only criteria for stopping the current motion set. For example, when the subject does not correctly perceive the motion applied, T_{w} can instead represent the total number of sequential wrong results on the feedback list, while T_{c} represents the total number of correct results on the feedback list.

In other embodiments, each of the motion sets 26 includes motion profiles that define motions of different types. For example, a motion set can have some motion profiles defining a pitch, while other motion profiles define a yaw. Or, a motion set can have motion profiles defining motions with different acceleration amplitudes A. Or, a motion set can have motion profiles defining motions with different frequencies f. In such embodiments, the criteria for creating and applying new motion sets to the subject to complete the vestibular test vary according to different motion profiles chosen for the motions within the same motion set.

In some embodiments, a coarse motion threshold A_{c} of an subject can be determined using the vestibular test of FIGS. 1-3 using relatively low thresholds τ_{w}, τ_{c}, and Δc. For example, τ_{w} equals 1, τ_{c} equals 2, and/or Δc is 25% of the last acceleration amplitude A. A follow-up vestibular test based on the coarse motion threshold A_{c} can then be used to determine a final motion threshold A_{f}. The follow-up vestibular test is conducted using the same test system disclosed in connection with FIGS. 1 and 2 but with motion sets that differ from those discussed in connection with FIG. 4, and methods that differ from those disclosed in connection with FIG. 3.

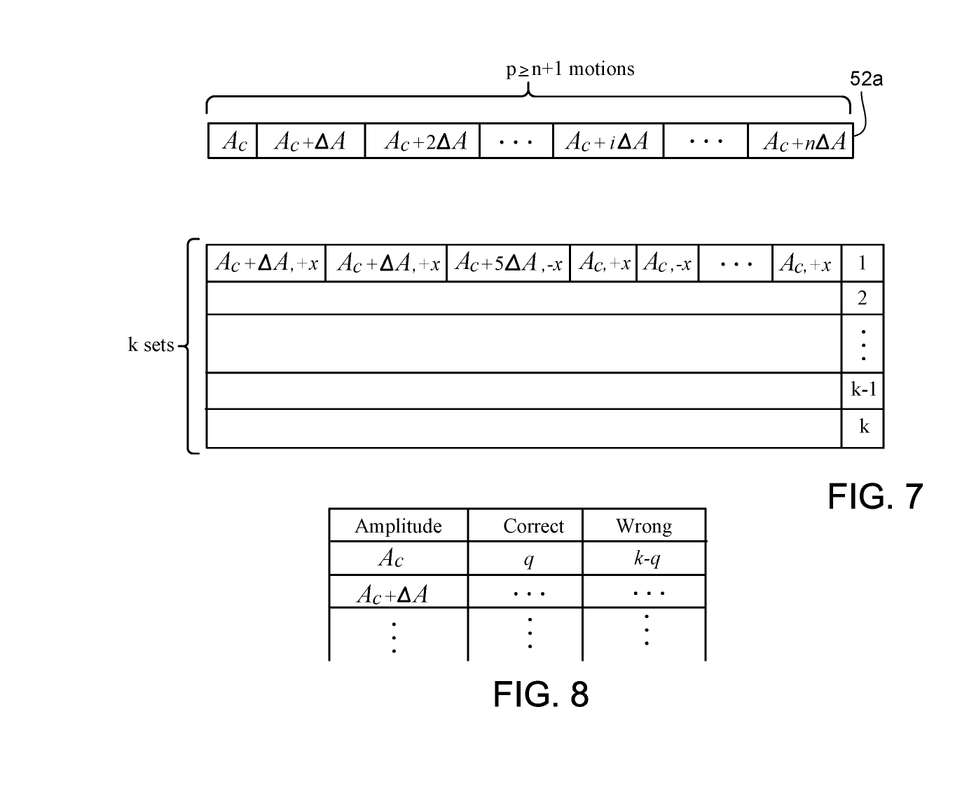

Referring to FIG. 7, the follow-up vestibular test applies, to the subject, a motion package 54 that includes k motion sets. Each of the k motion sets includes a total of p motions, each of which is characterized by a direction of the motion, e.g., “+x” or “−x” and an amplitude A of the motion. To generate each motion set, one first generates a standard motion set 52a having p motions without motion direction assignment. The motion amplitudes of the motions in the standard motion set 52a are chosen to define a range between A_{c} and A_{c}+nδA, where n is an integer and δA is an amplitude change, for example, 2% of A_{c}. Each amplitude can appear repeatedly within the standard motion set 52a so that p≥n+1. Each motion set, for example, motion set 52b, of the motion package 54 is generated by randomly selecting p motions from the sequence of the motions in the standard motion set 52a and randomly assigning a direction to each motion within each motion set.

The subject responds to each motion of the motion package 54. The number of right answers and wrong answers for each amplitude is then recorded in an answer table 56, as shown in FIG. 8. For example, the illustrated answer table 56 indicates that when a total number of k motions with amplitudes A_{c} was applied, the subject correctly perceived q of them and incorrectly perceived k-q of them. The statistical results in the answer table 56 can be analyzed using a generalized linear model or averaged normal cumulative distribution to yield a more refined estimate of the final motion threshold A_{f}. Detailed information for the generalized linear model is provided by McCullagh P, Nelder J A (1983), Generalized Linear Models.

In other embodiments, the vestibular test described in FIGS. 1-3 can be modified to be adapted for vestibulo-ocular reflex (VOR) analysis to use the vestibulo-ocular reflexes as an indicator of the subject's perception of motion. For example, the subject can be positioned on the motion platform 12 of FIG. 2 and be subjected to the motion tests of FIG. 3. However, instead of pushing buttons 19 (FIG. 2) at the end of each motion to indicate the perception of motion, the direction of the subject's reflexive eye movements can be measured and compared to the direction of the motion applied. The result of the comparison after each motion list is recorded on a feedback list similar to the feedback list 28 of FIG. 5 and analyzed using methods identical to those described earlier.

Having described the invention, and a preferred embodiment thereof,

We claim, as new and secured by Letters Patent:

1. An apparatus for determining a subject's threshold for perceiving acceleration, the apparatus comprising:
a motion platform to execute motions and to receive a response to the executed motions from a subject on the motion platform;
a feedback system in communication with the motion platform to receive the subject's responses, and to determine a next motion; the next motion having at least one feature determined based on the response of the subject to the executed motions; and
a controller connected to the motion platform and the feedback system to cause the motion platform to execute the motions defined by a motion set.

2. The apparatus of claim 1, wherein the controller is configured to cause the motion platform to execute a sinusoidal acceleration.

3. The apparatus of claim 1, wherein the motion set defines an acceleration having a maximum magnitude and the feature includes the maximum magnitude.

4. The apparatus of claim 1, wherein the feature includes a direction of the motion.

5. The apparatus of claim 1, wherein each motion belongs to a type selected from the group consisting of translation, rotation, and a combination of translation and rotation.

6. The apparatus of claim 1, wherein the feedback system detects the subject's vestibulo-ocular reflex.

7. A method for determining a subject's threshold for perceiving a motion, the method comprising:
(a) commencing execution of a first motion set defining a sequence of motions;
(b) recording responses of the subject to the motions in the sequence of motions defined by the first motion set;
(c) determining that the responses of the subject satisfy a condition;
(d) terminating execution of the first motion set;
(e) at least in part on the basis of the subject's responses to the motions in the sequence of motions defined by the first motion set, defining a second motion set defining a sequence of motions;
(f) commencing execution of the second motion set; and
(g) recording responses of the subject to the motions in the sequence of motions defined by the second motion set.

8. The method of claim 7, further comprising determining a maximum magnitude and frequency for each motion of the first motion set before commencing execution of a first motion set.

9. The method of claim 7, wherein determining that the responses of the subject satisfy a condition comprises determining how many recorded responses are correct and comparing the number of correct responses to a threshold.

10. The method of claim 7, wherein defining the second motion set comprises determining a maximum magnitude and frequency for each motion of the second motion set based on the recorded responses.

11. The method of claim 7, further comprising examining the responses of the subject to the motions defined by the first motion set and determining the subject's threshold before commencing execution of the second motion set.

12. The method of claim 7, further comprising, after step (g), defining the second motion set to be a new first motion set, and repeating the steps (c) to (d).

13. The method of claim 7, further comprising determining a motion threshold on the basis of the responses of the subject; selecting a new frequency; and repeating the steps (a) to (g) for determining thresholds corresponding to the new frequency.

14. The method of claim 7, wherein recording responses of the subject comprises observing the subject's vestibulo-ocular reflex.

15. A computer-readable medium having encoded thereon software for determining a subject's threshold for perceiving acceleration, the software including instructions for causing a data processing system to carry out the steps of
(a) commencing execution of a first motion set defining a sequence of motions;
(b) recording responses of the subject to the motions in the sequence of motions defined by the first motion set;
(c) determining that the responses of the subject satisfy a condition;
(d) terminating execution of the first motion set;
(e) at least in part on the basis of the subject's responses to the motions in the sequence of motions defined by the first motion set, defining a second motion set defining a sequence of motions;
(f) commencing execution of the second motion set; and
(g) recording responses of the subject to the motions in the sequence of motions defined by the second motion set.
